(19) United States
(12) Patent Application Publication
(10) Pub. No.: US 2024/0009592 A1
(43) Pub. Date: Jan. 11, 2024

(54) IMPROVEMENTS TO EXTRACTION METHODS, EXTRACTION SYSTEMS, COMPOUNDS AND FORMULATIONS

(71) Applicant: HEILALA VANILLA LIMITED, Hamilton (NZ)

(72) Inventors: John William VAN KLINK, Hamilton (NZ); Garth John BOGGISS, Hamilton (NZ)

(73) Assignee: HEILALA VANILLA LIMITED, Hamilton (NZ)

(21) Appl. No.: 18/252,972

(22) PCT Filed: Nov. 16, 2021
(86) PCT No.: PCT/NZ2021/050203

§ 371 Date: May 15, 2023

(30) Foreign Application Priority Data
Nov. 16, 2020 (NZ) 769969

(51) Int. Cl. B01D 11/02; A23L 27/10

(52) U.S. Cl. CPC B01D 11/0284; A23L 27/11

(57) ABSTRACT

Described are methods of extracting target compounds from vanilla, including compounds having beneficial anti-aging properties. The method includes the steps of performing a first extraction step on a feedstock to produce a first extract fraction and a marc; and a subsequent second extraction step on the marc to produce a second extract fraction. Also disclosed are formulations containing compounds containing active compounds obtained using the methods, and ingredients containing the actives compounds for use in various applications.

### 1. FIELD OF INVENTION

The present technology relates to improvements to extraction methods, extraction systems, compounds and formulations. It may find particular application in extracting lipid soluble compounds from natural products, compounds extracted using the methods and systems, and formulations containing the compounds.

### 2. BACKGROUND

Spices are used for flavouring foods and beverages, and in manufacturing cosmetics or perfumes. One widely used spice is vanilla, which is derived from orchids of the genus Vanilla. There are numerous 15 species of vanilla, but the species commonly cultivated commercially are Vanilla planifolia, Vanilla tahitensis, and Vanilla pompona; of these, Vanilla planifolia is the most important.

Vanilla is a relatively expensive spice to produce due to the significant labour involved in its cultivation. As a result, vanilla is a high value product.

What makes vanilla desirable for use as a spice is its distinctive flavour and aroma. This is a result of the complex mix of compounds which are extracted from the plant. Of these, vanillin (4-hydroxy-3-methoxybenazldehyde) is a major contributor to the characteristic flavour and aroma.

The compounds that provide vanilla with its distinctive flavour and aroma are present in the vanilla bean, which is the fruit produced by pollination of the vanilla flower. The vanilla beans are processed in various ways to produce a range of commercial products, including a whole vanilla bean, a powder (which comprises ground vanilla beans with other components), and a vanilla extract in the form of a vanillin and other compounds in a solvent e.g. ethanol.

However, commercial manufacture of vanilla extract produces significant amounts of waste products, being a marc of spent vanilla beans from which vanillin and other flavour compounds have been extracted. This waste stream is a cost point for vanilla manufacturers as it must be disposed of.

It is also known that vanilla beans contain active compounds that can have beneficial effects. For instance, WO 2007/034042 to Chanel Parfumes Beaute, describes that extracts from vanilla planifolia can be used in cosmetic and dermatological compositions to, inter alio, treat skin ageing.

The extracts comprise a lipid soluble fraction of compounds extracted from Vanilla planifolia. The fraction includes those compounds that are soluble in an oily phase following extraction of the fraction from the vanilla beans by an organic solvent. The preferred composition of the fraction is 0.5% to 10% of unsaturated monocarbonyl compounds, 20% to 80% of unsaturated dicarbonyl compounds, and 1% to 40% of unsaturated pyrannones.

WO 2007/034042 describes a process of extracting the lipid soluble fraction from raw (green) vanilla beans. The process involves preparing the raw (green) vanilla beans by milling and/or maceration, and a subsequent solvent extraction. Suitable solvents are describes as being a C₁-C₄ alcohol, or other organic solvents like propylene glycol, dipropylene glycol, ethyl acetate, hexane, or cyclohexane. Alternatively, it is suggested that supercritical fluid e.g. CO₂ extraction process can be used for this step.

The solution is filtered off from the vanilla beans, and the solvent removed to produce an oleoresin of vanilla. The oleoresin is subsequently subjected to a fractionation step e.g. gas chromatography or supercritical CO₂ to purify the oleoresin.

The purified oleoresin is subjected to a molecular distillation step, to isolate the desired oily distillate.

The process described in WO 2007/034042 extracts all compounds from the raw (green) vanilla bean, and then subsequently separates that into fractions of target compounds. The process does not extract compounds in a way that would allow them to be easily (and cost effectively) used as an ingredient in foodstuffs and beverages. As a result, the method of WO 2007/034042 produces a high value fraction for use in cosmetics, but it does not maximise the value returned from the vanilla beans through its use in products other than cosmetics.

There remains a need for improved methods and systems for extracting compounds from vanilla beans which balance the considerations of maximising product value, reducing manufacturing costs, and reducing waste streams.

Further, there is a need for improved cosmetic and dermatological formulations.

### 3. OBJECT OF THE TECHNOLOGY

It is an object of the technology to provide an improved extraction method and an improved extraction system.

Alternatively, it is an object to provide a method and system to derive value from a waste product of a commercial manufacturing process.

Alternatively, it is an object to provide at least one active compound, and methods and systems for extraction of the active compound(s).

Alternatively, it is an object to provide an extraction method and an extraction system which reduces use of consumables such as solvents in the extraction of at least one active compound.

Alternatively, it is an object to provide a method and system to extract at least one compound which has improved characteristics.

Alternatively, it is an object to provide a formulation containing at least one compound extracted using the methods and systems described herein.

Alternatively, it is an object to provide a formulation which has improved functional benefits.

Alternatively, it is an object of the invention to at least provide the public with a useful choice.

### 4. SUMMARY OF THE INVENTION

According to a first aspect of the technology, there is provided an extraction method, including the steps of:
(a) performing a first extraction step on a feedstock to produce a first extract fraction and a marc;
(b) subsequent to step (a) performing a second extraction step on the marc to produce a second extract fraction.

According to another aspect of the technology, there is provided an extraction method, including the step of extracting a target compound from a feedstock, wherein the feedstock is the fruit of a plant in the orchid family of the genus Vanilla, and further wherein the method uses a super critical CO₂ extraction process.

According to another aspect of the technology, there is provided an extraction system, wherein the system is configured to perform a method as substantially described herein.

According to another aspect of the technology, there is provided an active compound or mixture of active compounds manufactured according to a method as substantially described herein.

According to another aspect of the technology, there is provided a formulation containing at least one active compound manufactured according to a method as substantially described herein.

In one form, the feedstock may be the fruit of a plant in the orchid family of the genus Vanilla. In these forms, the feedstock may be vanilla beans, and reference will be made herein as such.

In a preferred form, the plant may be one or more of the species vanilla planifolia, vanilla tahitensis, or vanilla pompona, and the feedstock may be the beans of one or more of these species.

In particularly preferred forms, the feedstock may be “green” i.e. compounds have not yet been extracted from the raw material before it undergoes the first extraction step.

In a particularly preferred embodiment, the feedstock may be cured before it is used in the first extraction step e.g. the feedstock is a cured seedstock.

A cured feedstock for use with the present technology may comprises any curing process as should be known to one skilled in the art. For instance, a vanilla bean may be allowed to dry naturally on the plant before harvesting, or may be actively cured by known processes e.g. dipping, sweating, drying and conditioning.

However, it is also envisaged that the present technology may also use uncured vanilla beans or other feedstock Therefore, the discussion herein should not be seen as limiting.

In one form, the technology may involve a pre-treatment step.

Throughout the present specification, reference to the term “pre-treatment step” should be understood as meaning a process to prepare the feedstock which improves the efficiency or the accuracy of the first extraction process.

In a preferred form, the pre-treatment step may involve at least one of washing, drying, crushing, grinding, freezing, sizing by chopping, grinding and/or crushing.

In particularly preferred forms, the pre-treatment step does not remove a substantial amount of target compounds from the raw material before the first extraction step. As a result, the raw material can still be considered “green” when it undergoes the first extraction step.

Throughout the present specification, reference to the term “first extraction” should be understood as meaning a process to remove at least one compound or substance from the feedstock.

In a preferred form, the first extraction step may include a solvent extraction process.

In some forms, the solvent extraction process may use an organic solvent.

In particularly preferred forms, the solvent extraction process may use a food grade solvent. For instance, a suitable organic, food grade solvent for use with the present technology contains ethanol e.g. 35% by weight ethanol. The solvent may also be an aqueous solvent e.g. water, or a mixture of water and an alcohol such as ethanol. Alternatively, a suitable solvent may be glycerine.

In one form, the first extraction step may comprise one or more processes to assist with extraction of the compound(s) from the feedstock into the solvent. For instance, the first extraction step may comprise a heating step, enzyme assisted extraction process or ultrasonication process.

In a particularly preferred form, the one or more processes to assist with extraction of the compound(s) from the feedstock may occur concurrent with the first extraction step.

In a particularly preferred form, the first extraction step produces a first extract fraction.

Throughout the present specification, reference to the term “first extract fraction” should be understood as meaning the at least one compound or substance extracted by the first extraction step.

In a preferred form, the first extract fraction comprises one or more compounds which contribute to the flavour and aroma of vanilla.

In a particularly preferred form, the first extract fraction comprises vanillin.

In preferred forms, the first extract fraction may comprise a solution containing the at least one compound extracted by the first extraction step. For instance, the first extract fraction may comprise at least one of vanillin, vanillic acid (4-hydroxy-3-methoxybenzoic acid), p-hydroxybenzaldehyde and p-hydroxybenzoic acid.

In particularly preferred forms, the first extract fraction may comprise vanillin in an organic solvent e.g. an alcohol such as ethanol or glycerine, or a mixture of water and ethanol.

In addition, the first extract fraction may comprise one or more compounds that contribute to the flavour and aroma of vanilla. Accordingly, reference herein to the first extract fraction comprising vanillin should not be seen as limiting.

Throughout the present specification, reference to the term “marc” should be understood as meaning the feedstock after it has been subject to the first extraction step.

The marc may be substantially or completely spent of vanillin, and other compounds that contribute to the flavour and aroma of vanilla. However, the marc may still contain other compounds that are not extracted from the feedstock by the first extraction step.

Throughout the present specification, reference to the term “second extraction step” should be understood as meaning a process to remove at least one compound or substance from the marc.

In a particularly preferred form, the at least one compound or substance comprises a lipid soluble fraction. Reference herein will be made to at least one compound or substance as the lipid soluble fraction.

In further preferred forms, the lipid soluble fraction comprises at least one active compound e.g. which has a beneficial effect on skin aging.

In a preferred form, the second extraction step may involve a super critical CO₂ (“SCCO₂”) extraction process.

In a preferred form, the second extraction may remove a lipid soluble fraction from the marc i.e. the second extraction fraction comprises at least one lipid soluble compound.

It should be appreciated that the second extraction step produces a second marc, which is the feedstock after it has been subjected to the first extraction step and the second extraction step.

In some forms, the method of the present technology may include a third extraction step.

Throughout the present specification, reference to the term “third extraction step” should be understood as meaning a process to remove at least one compound or substance from the second marc.

In one form, the third extraction step may comprise involve a super critical CO₂ (“SCCO₂”) extraction process. For instance, the third extraction step may comprise a SCCO₂ process performed at different conditions to the second extraction step.

It is also envisaged that the third extraction process could involve different techniques. For instance, a solvent extraction process may be performed on the second marc.

In one form, the technology may include a preparation step.

Throughout the present specification, reference to the term “preparation step” should be understood as meaning processing or treating a marc to prepare it for a subsequent extraction process.

In a preferred form, the preparation step may involve at least one of drying, microbe reduction, separation, and size reduction.

In one form, the technology may comprise a post extraction step.

Throughout the present specification, reference to the term “post extraction step” should be understood as meaning a process to alter the second marc. For instance, the post extraction step may be used to transform the second marc into a product or component.

In one form, the post extraction step may involve at least one of washing, drying, grinding, and sizing. For example, the second marc (or third marc as the case may be) may be dried, ground and sized to produce a powder, which can be subsequently used as an ingredient or component of a formulation. In one form, the technology may comprise a fractionation step.

Throughout the present specification, reference to the term “fractionation step” should be understood as meaning a process to separate an extract into sub-fractions e.g. to separate the compounds in the extract from each other or into distinct mixtures of compounds.

In one form, the fractionation step may involve gas chromatography, molecular distillation or other suitable step as should be known to one skilled in the art.

Further aspects of the invention, which should be considered in all its novel aspects, will become apparent to those skilled in the art upon reading of the following description which provides at least one example of a practical application of the invention.

### 5. BRIEF DESCRIPTION OF THE DRAWINGS

One or more embodiments of the invention will be described below by way of example only, and without intending to be limiting, with reference to the following drawings, in which:

FIG. 1 Is a flow chart showing representative steps in a method according to one aspect of the present technology;

FIG. 2 Shows the extraction curve for Lab Scale Extraction 1;

FIG. 2B Shows the extraction curve for Lab Scale Extraction 2

FIG. 3 Shows the extract fractions obtained by Lab Scale Extraction 1;

FIG. 4 Shows the extract fractions obtained by Lab Scale Extraction 2;

FIG. 5 Shows a comparison of the marc and feed colour;

FIG. 6 Shows a comparison of the extraction curved for Lab Scale Extraction 2 and Lab Scale Extraction 3;

FIG. 7 Shows the extraction curve for the Commercial and Lab Scale Extractions;

FIG. 8 Shows a comparison of powder products produced by the technology;

FIG. 9 Shows a comparison of different packing densities between feed powder and extracted marc;

FIG. 10 Shows mean PCIP level in conditioned treatment groups.

FIG. 11 Shows selected analytical results of various samples produced by methods according to the present technology.

### 6. DETAILED DESCRIPTION OF THE PRESENT TECHNOLOGY

6.1 Overview of a Method according to the Present Technology

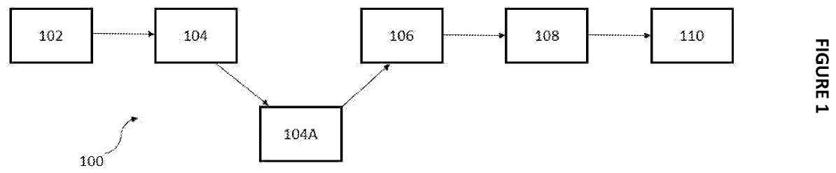

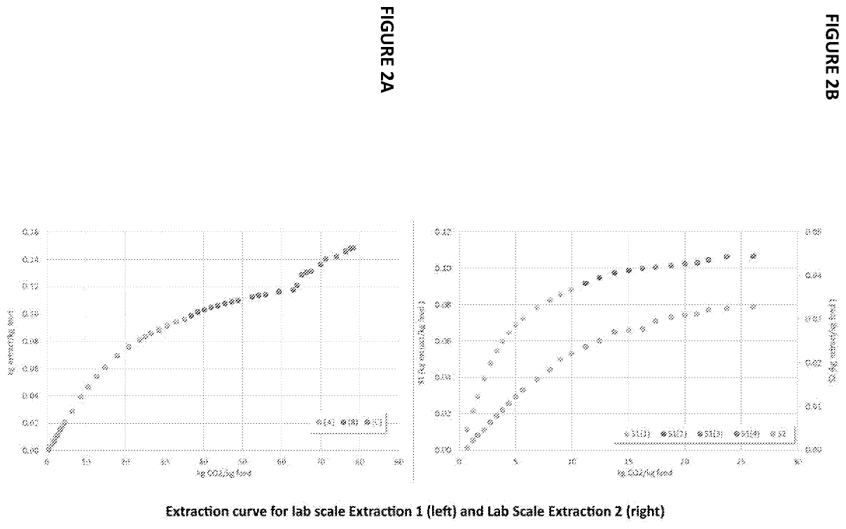

Referring first to FIG. 1 which shows representative steps in a method 100 according to one aspect of the technology.

The method 100 involves a pre-treatment step 102, a first extraction step 104, and a second extraction step 106.

In addition, the method includes a preparation step 104A, a post extraction step 108 and a fractionation step 110.

The method 100 is configured to selectively extract one or more compounds from a feedstock, which in 30 the preferred form comprises vanilla beans (not shown in the Figures). The vanilla beans can be the fruit of any known variety of vanilla, e.g. vanilla planafolia. Further aspects of the method 100 should become clearer from the following discussion.

### 6.1.1 Pre-Treatment Step

During the pre-treatment step, raw (green) vanilla beans are subjected to one or more processes to assist with increasing the efficiency of the first extraction step 104 which is to follow. Suitable techniques for the pre-treatment step include at least one of grinding, maceration and sizing.

The raw (green) vanilla beans may have been cured by techniques as should be known to one skilled I the art before the pre-treatment step. Alternatively,

### 6.1.2 First Extraction Step

The first extraction step 104 is used to produce a first extract fraction comprising one or more compounds having a desired taste or flavour profile. For instance, the target compounds comprise a mixture containing vanillin and optionally one or more other compounds.

Any suitable process may be used for the first extraction step 104. However, in the preferred embodiment, the first extraction process is a solvent extraction as should be known to one skilled in the art.

Suitable solvents include ethanol, a mixture of water and ethanol methanol, acetonitrile, acetone, chloroform and hexane, or any other solvent in which vanillin is soluble. However, in the preferred form, the solvent is ethanol e.g. at least 35% w/w or other food or cosmetic grade solvent e.g. glycerine.

To perform the first extraction step 104, the solvent and the vanilla beans are mixed together for a pre-determined period of time.

In addition, other techniques may used to improve extraction of the target compounds from the vanilla beans, as should be known to one skilled in the art. For instance, the first extraction step may be an assisted solvent extraction process using techniques such as heating agitation/stirring, or ultrasonication.

After the pre-determined period of time, the solvent is separated from the vanilla beans e.g. by filtration or decanting. Separation of the solvent and vanilla beans produces a marc (not illustrated in the Figures), being the vanilla beans which are at least partially, substantially or completely spent of the target compound e.g. vanillin and one or more compounds that contribute to the flavour and aroma of vanilla.

The first extraction step 104 produces a first extraction fraction, which comprises a vanilla extract e.g. vanillin (and optionally one or more other compounds), in the solvent.

### 6.1.3 Preparation Step

The method 100 optionally includes a preparation step 104A. In the preparation step 104A, the marc (not illustrated in the Figures) produced by the first extraction step 104 is prepared before being used in the second extraction step 106.

The preparation step 104A may involve one or more of the following processes:
1. microbe reduction;
2. drying;
3. separation e.g. to remove vanilla seeds from the vanilla beans;
4. size reduction e.g. a grinding process such as coarse mill.

The methods and components used to complete the process(es) of the preparation step 104A are as should be known to one skilled in the art. Further aspects of the preparation step 104A should become clearer from the following description.

6.1.4 Second Extraction Step

The method 100 includes a second extraction step 106. The second extraction step comprises a process to extract compounds from the marc produced by the first extraction step 104. The second extraction step 106 produces a second extract fraction which contains one or more target compounds e.g. a mixture of lipid soluble compounds. The second extract fraction therefore comprises the lipid soluble fraction.

The second extraction step 106 may comprise any known method or system for extracting target components. However, in the preferred form, the second extraction step 106 comprises super critical CO₂ (SCCO₂) extraction and reference will be made herein as such.

The conditions and duration of the second extraction step 106 can be varied to achieve a desired composition for the second extract fraction. For instance, the time, pressure, flow rate, temperature and feed ratio may all be varied.

In some forms, the second extraction step 106 may be performed in multiple steps e.g. it also involves a third extraction step 11013, and a fourth extraction step 110C. The third extraction step 11013 and the fourth extraction step 110C differ to each other in the parameters under which the extraction occurs. For instance, at least one of the time, pressure, flow rate, temperature and feed ratio may all be varied to achieve a desired extract profile.

### 6.1.5 Post Extraction Step

The post extraction step 108 can be performed on the second marc produced by the second extraction step 106.

For instance, the post extraction step 106 can be used to convert the second marc into a commercial product. In these embodiments, the post extraction may produce a powder suitable for use as an ingredient in food or cosmetics. The second extraction step may involve at least one of washing, drying, grinding, and sizing.

### 6.1.6 Fractionation Step

The method 100 optionally includes a fractionation step 110 which can be used to separate the second extract fraction into mixtures of compounds or substantially purified compounds. For instance, the fractionation step 110 may comprise gas chromatography to produce two or more distinct fractions of active compounds.

### 6.2 Extraction Examples

Further features of the method 100 should become clearer from the following discussion of examples of the technology which are provided in non-limiting terms and do not narrow the scope of the technology.

### 6.2.1 Lab Scale Extraction 1

A marc was prepared by performing a first extraction step 104 as described above. The marc was subsequently processed by preparation step 104A, to produce a dried, ground marc powder.

800 g of the dried, ground marc powder was placed in a 2 L extraction vessel with sintered filter discs at both ends, filling the vessel completely with a packing density of approximately 0.4 g/mL. The vessel was then pressurised with CO₂ and the extraction was started. The extraction was carried out in three steps and the three extracts obtained were kept separate. The first step (extract A) was carried out at 120 bar and 40° C. until a 35:1 CO₂ to feed ratio had been circulated; the plant was then boxed in overnight and the extraction was carried on the next day under the same conditions until an additional 28:1 CO₂ to feed ratio had been circulated (extract 6); at this point the pressure and temperature were increased to 400 bar and 50° C. respectively and the third and last step of the extraction was carried on until an additional 15:1 CO₂ to feed ratio had been circulated (extract C). The final CO₂ to feed ratio was 78:1. Throughout these steps, the solvent containing the dissolved extract after passing through the bed was depressurized and passed into a separation vessel where the extract was accumulated and gas phase CO₂ from the separator was condensed and recirculated. Extract accumulating in the separation vessel was recovered through a valve periodically during the run to determine the progress of the extraction. After extraction, the plant was depressurized and the residual marc was allowed to degas before being unloaded. Extraction parameters are listed in Table 1. An ethanol wash was applied to the plant following the run to estimate the amount of residual extract that remained in the lines.

TABLE 1
Extraction conditions (extraction 1)

| | Step 1 | Step 2 | Step 3 |
|---|---|---|---|
| Feed Mass (g) | 800 | | |
| Extraction pressure (bar) | 120 | 120 | 400 |
| Extraction temperature (° C.) | 40 | 40 | 50 |
| Separator pressure (bar) | 56 | 55 | 51 |
| Separator temperature (C) | 40 | 40 | 50 |
| Average CO2 flow rate | 2.8 | 2.8 | 3.2 |
| CO₂ to feed ratio | 35:1 | 28:1 | 15:1 |

### 6.2.2 Lab Scale Extraction 2

A marc was prepared by performing a first extraction step 104 as described above. The marc was subsequently processed by preparation step 104A, to produce a dried, ground marc powder. 800 g of the dried, ground marc powder was placed in a 2 L extraction vessel with sintered filter discs at both ends, filling the vessel completely with a packing density of approximately 0.4 g/mL. The vessel was then pressurised with CO₂ and the extraction was started. The extraction was initially carried out at 300 bar and 40° C., and the pressure was increased to 450 bar after a 21:1 CO₂ to feed ratio had been circulated. The CO₂ containing the dissolved extract was first depressurized down to 90 bar at 40° C. and passed into a first separation vessel where the first (least volatile) extract fraction, S1, was accumulated. Following this, the CO₂ phase was further depressurized through a second valve to approximately 54 bar and 40° C. where the second (more volatile) extract fraction, S2, was accumulated. Gas phase CO₂ from the second separator was condensed and recirculated. Extract accumulating in the separation vessels was recovered through a valve periodically during the run to determine the progress of the extraction. After extraction, the plant was depressurized and the residual marc was allowed to degas before being unloaded. Extraction parameters are listed in Table 1. The final CO₂ to feed ratio was 26:1. The first extract fraction, S1, was fractionated into 4 separate extracts, collected at different CO₂ to feed ratios: 51(1) from 0 to 10:1, S1(2) from 10:1 to 15:1, S1(3) from 15:1 to 20:1, and S1(4) from 20:1 to the end (corresponding with an increase in extraction pressure). The second extract fraction, S2, was collected throughout the run and not fractionated. An ethanol wash was applied to the plant following the run to estimate the amount of residual extract that remained in the lines.

TABLE 2
Extraction conditions (Lab Scale Extraction 2)

| | S1(1) | S1(2) | S1(3) | S1(4) | S2 |
|---|---|---|---|---|---|
| Feed Mass (g) | 800 | | | | |
| Extraction pressure (bar) | 300 | 300 | 300 | 450 | 300-450 |
| Extraction temperature (° C.) | 40 | 40 | 40 | 40 | 40 |
| Separator 1 pressure (bar) | 90 | 90 | 90 | 90 | — |
| Separator 1 temperature | 40 | 40 | 40 | 40 | — |
| Separator 2 pressure (bar) | — | — | — | — | 54 |
| Separator 2 temperature | — | — | — | — | 40 |
| Average CO₂ flow rate (kg/h) | 2.6 | 2.8 | 3.1 | 3.5 | 2.9 |
| CO₂ to feed ratio | 10:1 | 5:1 | 5:1 | 6:1 | 26:1 |

### 6.2.3 Lab Scale Extraction 3

An additional lab scale extraction was carried out, aiming to assess the colour retention of the marc as well as the effect of particle size on the extraction yield. The feed material was milled prior to the extraction using a Wiley knife mill and a 2 mm mesh attached, and the extraction was carried out using the same conditions as the Lab Scale Extraction 2 discussed in section 6.2.2 above, but with a shortened duration (10:1 CO₂:feed) to avoid extraction of dark coloured compounds.

TABLE 3
Extraction conditions (Lab Scale Extraction 3)

| | |
|---|---|
| Feed Mass (g) | 365 |
| Extraction pressure (bar) | 300 |
| Extraction temperature (° C.) | 40 |
| Separator 1 pressure (bar) | 90 |
| Separator 1 temperature | 40 |
| Separator 2 pressure (bar) | 52 |
| Separator 2 temperature | 40 |
| Average CO₂ flow rate (kg/h) | 2.0 |
| CO₂ to feed ratio | 10:1 |

### 6.2.4 Commercial Scale Extraction

A marc was prepared by performing a first extraction step 104 as described above. The marc was subsequently processed by preparation step 104A, to produce a dried, ground marc.

360 kg of the dried, ground marc was extracted using Pharmalink's manufacturing plant (3 by 850 L capacity). The beans were extracted with supercritical CO2 at 300 bar and 40° C. using a single 850 L vessel, with a final solvent to feed ratio of 40:1 (i.e. 40 kg of CO₂ were circulated per kg of beans). The extraction was carried out over a 4 hour period. The extract was fractionated into a first separator fraction (i.e. S401, 90 bar and 40° C.) and a second separator fraction (i.e. S403, approximately 45 bar and 40° C.). The first separator fraction was further fractionated into two separate fractions: the first one (interim) was collected after a 10:1 CO₂:feed had been circulated, and the second one (final) was collected at the end of the run. The second separator fraction was collected at the end of the run and the water present in this fraction was decanted off and kept separate. Samples of feed, marc and all extract fractions were sent to PFR for analysis.

### 6.3 Results and Discussion of Extraction Examples

### 6.3.1 Lab Scale Extractions

The total extraction yield obtained in Lab Scale Extraction 1 and Lab Scale Extraction 2 were relatively similar (Table 4), with Lab Scale Extraction 1 being slightly higher possibly due to the longer overall extraction time and higher extraction pressure used in the final step. In Lab Scale Extraction 2, the pressure was also increased to 450 bar at the final step but the total extraction time was shorter than for Lab Scale Extraction 1.

The extraction curves (FIGS. 2A and 2B) show a constant rate extraction for approximately the first 4% extract yield, after which the extraction becomes slower—limited by diffusion rates.

TABLE 4
Extraction yields

| | | Wet basis | Dry basis |
|---|---|---|---|
| Extraction | Extract A | 9.6% | 9.7% |
| 1 | Extract B | 2.1% | 2.2% |
| | Extract C | 3.1% | 3.3% |
| | Total Yield | 14.9% | 15.2% |
| Extraction | S1(1) | 8.8% | 9.4% |
| 2 | S1(2) | 1.0% | 1.1% |
| | S1(3) | 0.4% | 0.4% |
| | S1(4) | 0.6% | 0.6% |
| | S2 | 3.5% | 2.9% |
| | Total Yield | 14.3% | 14.4% |
| Extraction | S1 | 9.7% | 10.3% |
| 3 | S2 | 2.5% | 2.7% |
| | Total Yield | 12.2% | 13.0% |

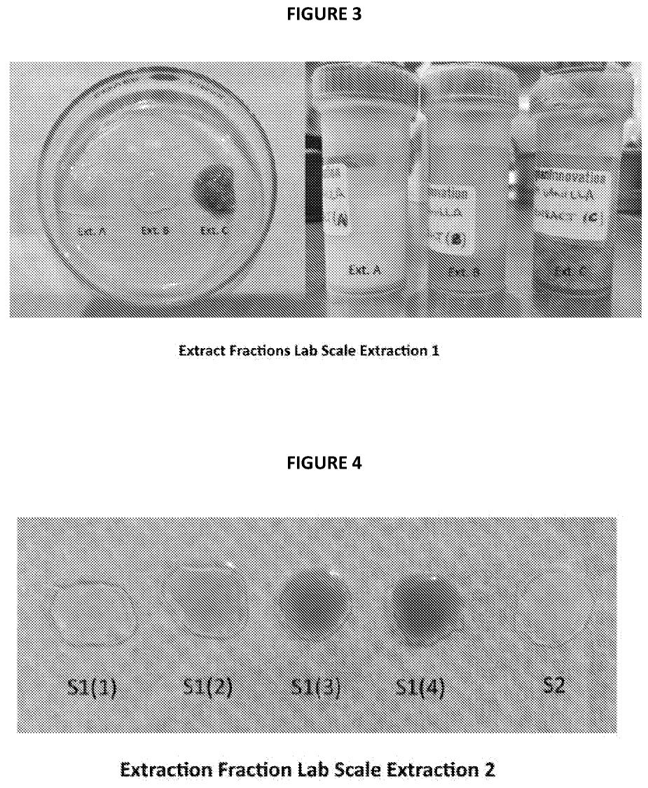

The extract obtained in Lab Scale Extraction 1 was initially pale yellow with an appearance very similar to whipped butter, but as the extraction progressed the colour gradually darkened, and turned green when the pressure was increased to 400 bar (FIG. 3). Similarly, in Lab Scale Extraction 2, the 51 fractions were initially pale yellow but the colour intensity and the viscosity increased gradually with extraction time as the less soluble colour compounds and waxes were extracted (FIG. 4). A green tinge is already visible in S1(2), and this gets darker in later fractions. The S2 fraction maintained a pale yellow colour throughout the run.

The moisture content of the feed was measured at 5.85% 1, and a small amount of water was co-extracted in both extractions. In extraction 1, 3.7 g free water were decanted off extract A (4.8% of the total extract A mass), and 0.67 g were recovered from extract B (3.9% of total extract B mass). In extraction 2, water was collected in S2, and 5.9 g free water was decanted (21.3% of S2 mass). The S2 fraction is noticeably more fragrant with a characteristic Vanilla aroma.

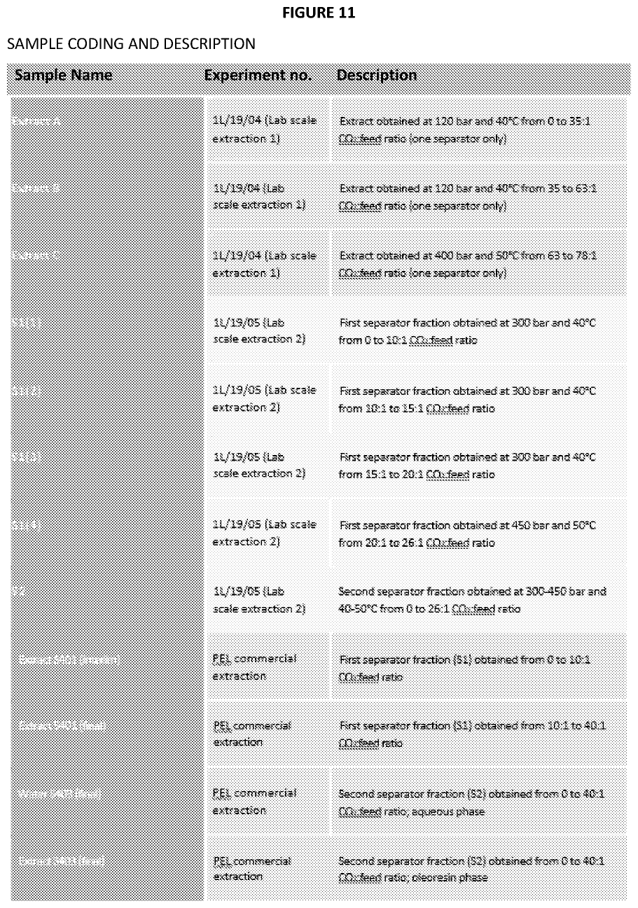

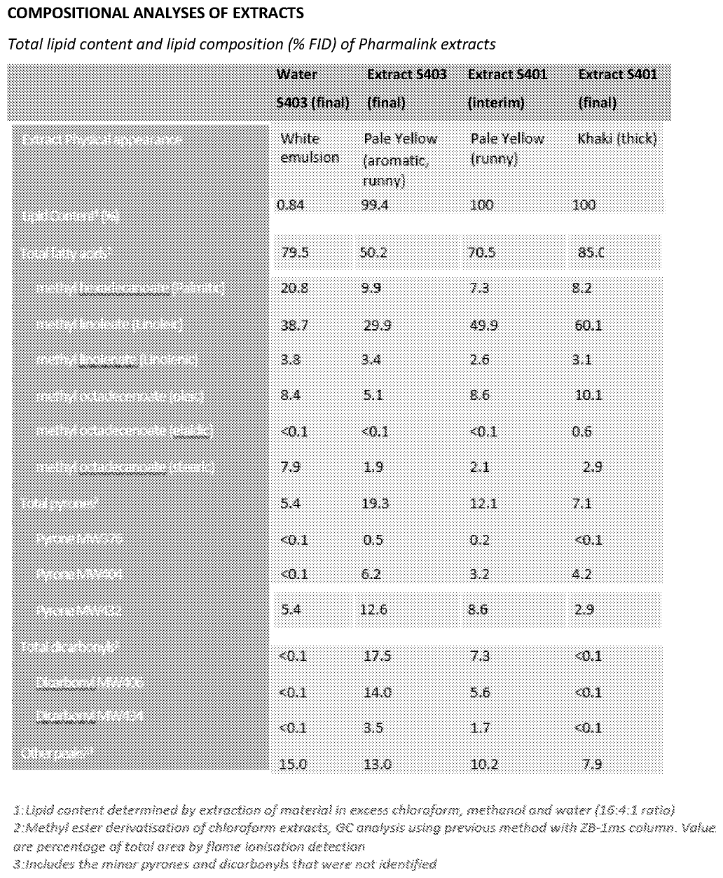

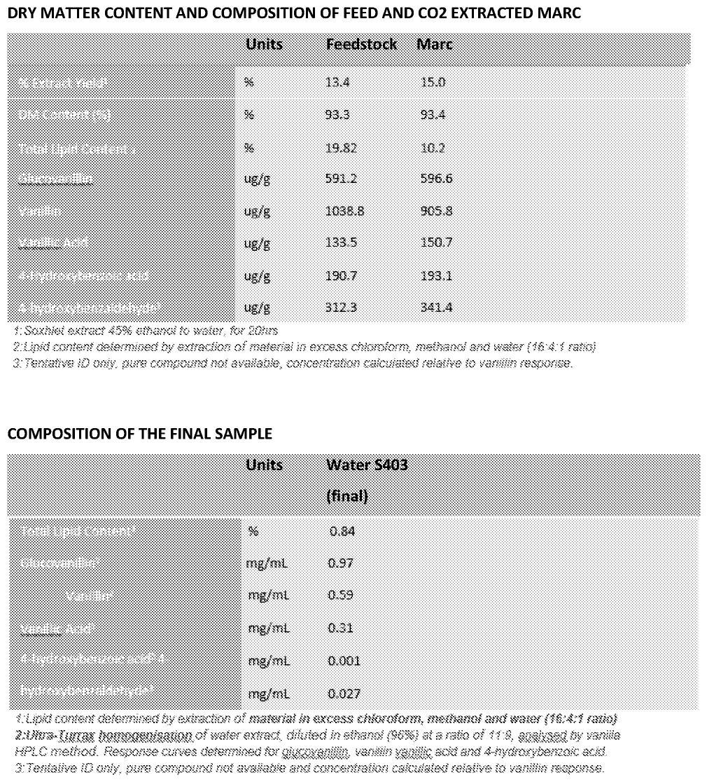

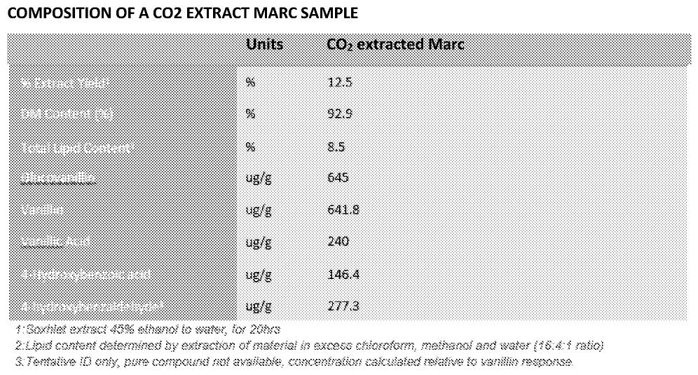

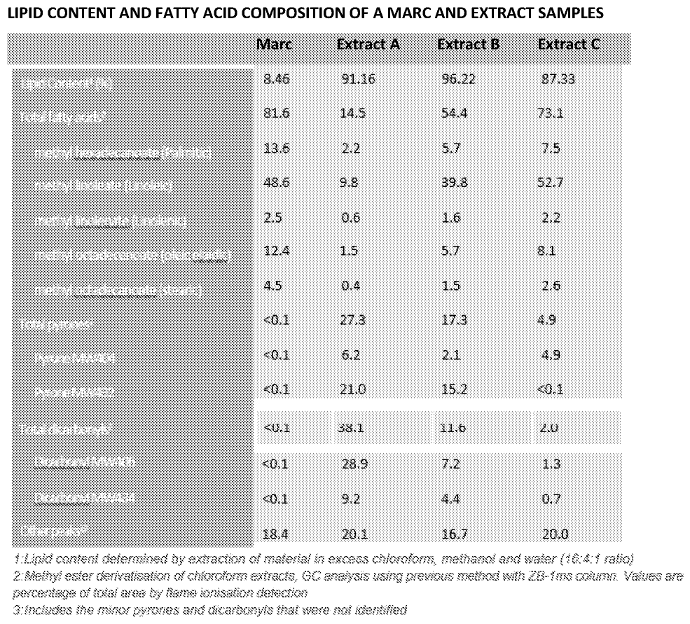

The analytical results from Lab Scale Extraction 1 (see FIG. 11) indicated a relatively high lipid content in the marc (8.5%). This could be caused by the presence of polar lipids that are not able to be extracted by CO₂, or to lipids that are strongly bound. The lipid mass balance (i.e. the lipids present in the extracts and marc relative to the lipids present in the feed) is 102.0%. Pyrones and dicarbonyl compounds are efficiently extracted early on, with minimal residual levels present in the marc and the highest levels in extract A.

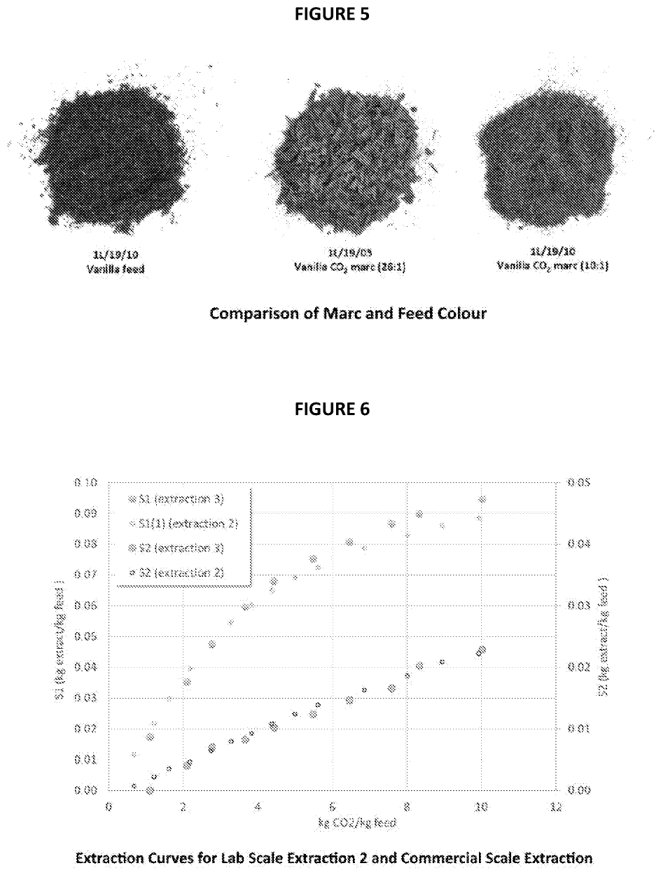

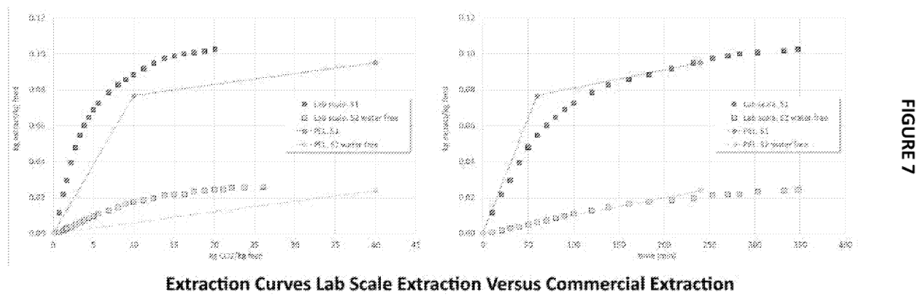

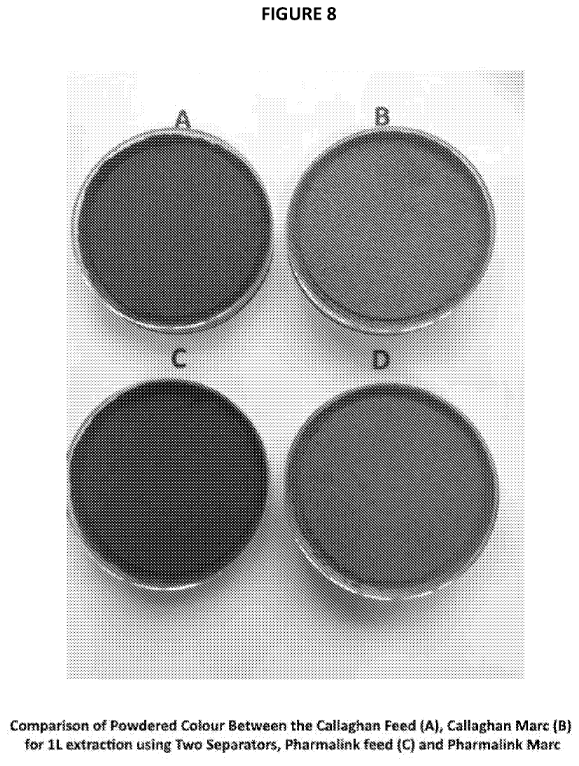

As seen in FIG. 5, the marc obtained in extraction 3 (right) was slightly darker than the one obtained in the extraction 2 after a 26:1 CO₂:feed ratio (middle), but still visibly lighter than the feed (left). The total extraction yield obtained in this extraction was 12.2% (wet basis). The yield from the first separator was slightly higher than the yield obtained for the same extraction period in Lab Scale Extraction 2 (9.7% vs 8.8%). The extraction curve for S1 (FIG. 6) is equivalent to the one obtained in Lab Scale Extraction 2 during the first, solubility limited stage of the extraction (i.e. until about 4% yield), and after this point Commercial Scale Extraction proceeded slightly faster than Lab Scale extraction 2, achieving a higher yield at the 10:1 point. This is attributed to the smaller particle size used in Commercial Extraction, which would improve the extraction on the diffusion limited stage (i.e. after about 4% yield). The yield from the second separator was equivalent to the one obtained in Lab Scale extraction 2 for the same extraction period, and only a small amount of water was observed in S2 (too small to accurately separate from the oil phase).

### 6.3.2 Commercial Scale Extraction

The total extraction yield obtained in the extraction carried out at Pharmalink was 11.9% (13.0% including the water phase). A comparison between the lab and commercial extraction curves is shown in FIG. 7. In Lab Scale Extraction 2, only the 51 yield obtained at 300 bar is shown (i.e. excluding S1(4)).

The total 51 yield obtained in the commercial extraction was slightly lower (9.5% vs 10.3%), even though the extraction was run for a longer solvent:feed ratio (FIG. 7); this is largely caused by the higher effective solvent flow rate used in the commercial extraction which results in a shorter extraction period. This effect can be seen in FIG. 7 which shows the extraction on a total extraction time basis.

The analytical results for the commercial scale extraction (see Appendix) show a 10.2% residual lipid content in the marc, which is consistent with the lab scale findings. The lipid mass balance is 104.8% (note that this was obtained with an estimated value of the marc, calculated by mass difference between the feed and the extract fractions).

TABLE 5
Commercial extraction yields

| | | S1 | | | |
|---|---|---|---|---|---|
| | | Interim | Final | S2 | |
| | Feed | (0-10:1) | (10:1-40:1) | Product | Water |
| Mass (kg) | 360.5 | 27.67 | 6.64 | 8.6 | 3.9 |
| Yield | — | 7.7% | 1.8% | 2.4% | 1.1% |

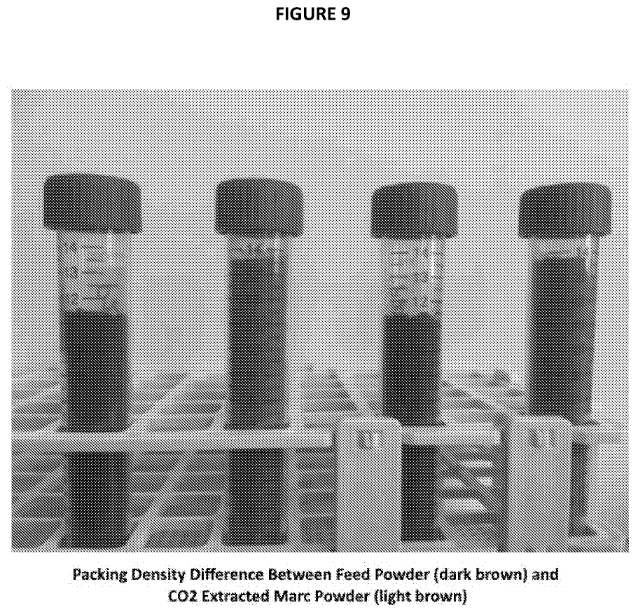

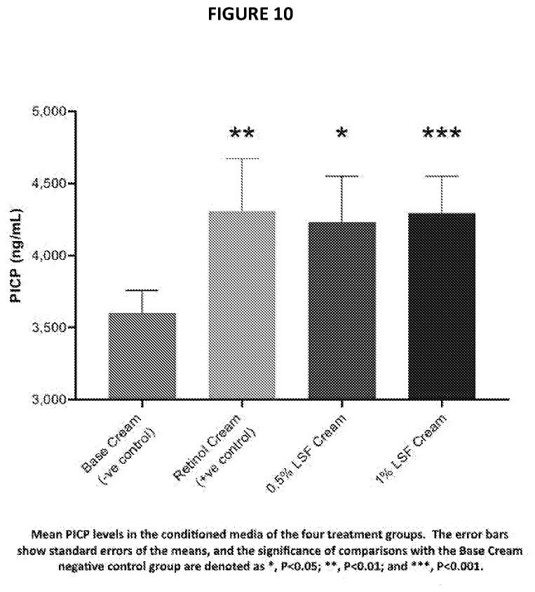

Subsamples (3.6 g) of the marc produced by Lab Scale Extraction 1 and the Commercial Scale Up were milled through 0.25 mm screen (Retsch ZM 100 mill) to a fine powder and photographed under uniform light conditions (an are shown as FIG. 9). As well as the difference in colour it was noted the marc powders had a lower packing density compared to the feed powders (as is shown in FIG. 10).

### 6.4 Efficacy Investigations

### 6.4.1 Introduction

The activity of a lipid-soluble extract of vanilla (ISF) manufactured according to the method 100 on beneficial collagen synthesis was investigated.

### 6.4.2 Objective

The aim of the investigation was to evaluate the effect of the LSF on the synthesis of collagen type I in vitro using the full thickness human 3D skin model EpiDermFT.¹ ¹(MatTek Corporation) [1], which has been widely used for a wide range of applications including anti-aging studies [2,3]

### 6.4.3 Methods

EpiDerm FT tissues were randomly allocated to groups (N=4 tissues/group) and were treated with 0.5% LSF in base cream or 1% LSF in base cream. Two further groups of tissues (N=4 each) were treated with just the base cream vehicle (negative control group), or with 0.5% retinol in base cream (positive control group [4,5]). Following administration of 100 μL of each cream treatment the tissues were cultured for three hours and the creams were then rinsed off using sterile phosphate-buffered saline (PBS). The tissues were cultured overnight for a further 21 hours, after which the conditioned cell culture media were collected for determination of procollagen I C-peptide (PICP). PICP is a marker of fresh collagen synthesis and was measured using an EIA kit (TaKaRa) following the manufacturer's instructions.

### 6.4.4 Results

The mean levels of PICP measured in the conditioned media of each treatment group are shown in FIG. 11. PICP levels were significantly elevated by 18-20% in the media of the 0.5% retinol cream group (positive control) and both concentrations of LSF cream as compared to the base cream (negative control). There were no significant differences in the PICP levels of the three positively responding groups.

### 6.4.5 Conclusions

The LSF increased the production of PICP, and thus collagen type I, by the EpiDermFT full-thickness human skin model tissues.
0.5% LSF in base cream was as effective as 1% LSF.
Both LSF treatments were as effective as the 0.5% retinol cream positive control treatment.
The results demonstrate that the LSF could be an effective anti-aging ingredient for cosmetic formulations.

### 7. COSMETIC FORMULATION

The lipid soluble fraction obtained according to the method 100 may be sold as either an ingredient for use in subsequent cosmetic formulation e.g. to manufacturers of cosmetics, or formulated into a cosmetic formulation and sold to retailers or consumers.

A representative formulation for a cosmetic according to the technology is summarised in table 6 below,

TABLE 6
Representative formulation of a cosmetic according to the present invention.

| Part | Trade name | INCI Description | Supplier | % wt |
|---|---|---|---|---|
| A | Marula oil | Sclerocarya Birrea (Marula) Seed Oil | Pure Ingredients | 4.00 |
| | Kakaduplum oil | Terminalia Ferdinandiana (Kakadu | Pure Ingredients | 4.00 |
| | | plum) seed oil | | |
| | Safflower oil | Carthamus Tinctorius (Safflower) | Pure Ingredients | 4.00 |
| | | Seed Oil | | |
| | Lipid soluble fraction | Vanilla Planifolia (Vanilla) Fruit | Heilala Vanilla | 0.50 |
| | | Extract | | |
| | Vitamin E natural | Tocopherol | various | 1.00 |
| | Avocado oil | Persea gratissima (Avocado) Oil | various | 1.00 |
| | Jojoba oil | Simmondsia Chinensis (Jojoba) Seed | Pure Ingredients | 69.70 |
| | | Oil | | |
| | Rosehip oil | Rosa Canina Fruit Oil | Pure Ingredients | 4.00 |
| | L 22 | Jojoba Oil/Macadamia Seed Oil Esters | Verital | 2.50 |
| | | (and) Squalene (and) Phytosteryl | | |
| | | Macadamiate (and) Phytosterols | | |
| | | (and) Tocopherol | | |
| | Sytenol A | Bakuchiol | DKSH | 0.80 |
| | Oleosoft-4 | OLIVE GLYCERIDES, ALMOND | Connell | 5.00 |
| | | GLYCERIDES, LINSEED GLYCERIDES, | Australasia | |
| | | BORAGE SEED GLYCERIDES, | | |
| | | TOCOPHERYL ACETATE | | |
| | Nikko VC IPV | Ascorbyl Tetraisopalmitate | CeeChem | 2.50 |
| | Vitamin C | | | |
| | TBC | Fragrance | Innovaction | 1.00 |

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

The invention may also be said broadly to consist in the parts, elements, characteristics and features referred to or indicated in the specification of the application, individually or collectively, in any or all combinations of two or more of said parts, elements, characteristics or features.

Aspects of the present invention have been described by way of example only and it should be appreciated that modifications and additions may be made thereto without departing from the scope thereof as defined herein.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

1-93. (canceled)

94. An extraction method comprising:
(a) performing a first extraction step on a feedstock to produce a first extract fraction and a marc; and
(b) subsequent to step (a) performing a second extraction step on the marc to produce a second extract fraction.

95. The method of claim 94, wherein the feedstock is vanilla beans.

96. The method of claim 94, wherein the feedstock is a raw material.

97. The method of claim 94, wherein the first extraction step does not substantially or completely extract target compounds to be extracted at the second extraction step.

98. The method of claim 94, wherein the marc is substantially or completely spent of compounds that contribute to the flavour and aroma of vanilla.

99. The method of claim 94, wherein the second extraction step comprises extracting a lipid soluble fraction from the marc.

100. The method of claim 99, wherein the lipid soluble fraction comprises at least one active compound that has a beneficial effect on skin aging.

101. The method of claim 94, wherein the second extraction step involves a super critical CO₂ (“SCCO₂”) extraction process.

102. The method of claim 94, further comprising a third extraction step.

103. The method of claim 102, wherein the third extraction step involves a process to remove at least one compound or substance from the second marc.

104. The method of claim 103, wherein the third extraction step comprises an SCCO₂ extraction process performed at different conditions to a SCCO₂ performed at the second extraction step.

105. An extraction method, including a step of extracting a target compound from a feedstock, wherein the feedstock is the fruit of a plant in the orchid family of the genus Vanilla, and further wherein the method utilizes an SCCO₂ extraction process.

106. The method of claim 105, wherein the method further includes a first extraction step which occurs before the SCCO₂ process wherein the first extraction step includes a food grade solvent extraction process and wherein the food grade solvent contains substantially 35% by weight ethanol.

107. The method of claim 105, wherein the method further includes a first extraction step which occurs before the SCCO₂ extraction process and wherein the first extraction step produces a marc wherein the marc is substantially or completely spent of compounds that contribute to the flavour and aroma of vanilla.

108. The method of claim 107, wherein the SCCO₂ extraction process comprises extracting a lipid soluble fraction from the marc.

109. The method of claim 108, wherein the lipid soluble fraction comprises at least one active compound.

110. The method of claim 109, wherein the at least one active compound has a beneficial effect on skin aging.

111. The method of claim 105, wherein the SCCO₂ extraction process produces a second marc and wherein the method further comprises an extraction process to remove at least one compound or substance from the second marc.

112. The method of claim 111, wherein the extraction process to remove at least one compound or substance from the second marc comprises an SCCO₂ process performed at different conditions to the SCCO₂ extraction process to produce the first marc.

113. A formulation comprising at least one active compound extracted by the method of claim 94.
